(19) United States
(12) Patent Application Publication
(10) Pub. No.: US 2006/0122519 A1
(43) Pub. Date: Jun. 8, 2006

(54) Heartbeat measuring device

(76) Inventor: Hisao Nakamura, Chiba-shi (JP)

Correspondence Address: BRUCE L. ADAMS, ESQ.;SUITE 1231, 17 BATTERY PLACE, NEW YORK, NY, 10004, US

(21) Appl. No.: 11/288,732

(22) Filed: Nov. 29, 2005

(30) Foreign Application Priority Data
Dec. 8, 2004 (JP) 2004-355513

(51) Int. Cl. A61B 5/02

(52) U.S. Cl. 600/502; 206/811

(57) ABSTRACT

To make it possible to simply and certainly perform a check of a waterproofness of a heartbeat measuring device not only before a product shipment but also at the time when a user uses it. In a heartbeat measuring device having a case and a case back which are made of a opaque hard plastic, a circuit board accommodated in an accommodation chamber formed by the case and the case back, various electronic parts attached to the circuit board, and an antenna, a through-hole is provided in a center part of the opaque case, and a transparent member is liquid-tightly fixed to an outside opening part of the through-hole, thereby making it possible to check the waterproofness of the heartbeat measuring device by observing a dew condensation generated on an inner face of the transparent member.

### BACKGROUND OF THE INVENTION

1. Field of the Invention

The present invention relates to a heartbeat measuring device in which a heartbeat measuring circuit and the like are accommodated in an opaque waterproof case, and which is mounted to a user's chest when used, and relates especially to its structure of a waterproofness check.

2. Description of the Prior Art

As disclosed in JP-A-10-155753 Gazette for instance, the heartbeat measuring device is an electronic device in which device constitution parts such as heartbeat measuring circuit, transmitting circuit, control circuit, antenna and battery are accommodated in the opaque waterproof case. A heartbeat data that the heartbeat measuring circuit of the heartbeat measuring device has measured is radio-transmitted to a heartbeat number display through the transmitting circuit and the antenna. The heartbeat number display is the electronic device having the antenna, a receiving circuit, a liquid crystal display part, the control circuit and the battery, and is a wristwatch type display mounted to a user's wrist for instance. Accordingly, the user can simply grasp his/her own heartbeat number during the use. Since the heartbeat measuring device is one used by being mounted to the user's chest, moisture such as sweat contacts directly with the heartbeat measuring device. Accordingly, in the conventional heartbeat measuring device, there is adopted such a waterproof structure that a case back is fixed to the case through a packing.

That is, as shown in a bottom view of FIG. 3 shown with the case back being removed and a sectional view of FIG. 4 shown by being cut in a longitudinal direction, the conventional heartbeat measuring device has a case 1 and a case back 2 which are made of an opaque hard plastic, a circuit board 6 accommodated in an accommodation chamber formed by the case 1 and the case back 2, various electronic parts 7 attached to the circuit board 6, and an antenna 8. The circuit board 6 and the various electronic parts 7 constitute the heartbeat measuring circuit, the transmitting circuit and the control circuit. A battery 9 is accommodated in a battery accommodation chamber formed in the case back 2. An opening of the battery accommodation chamber is sealed by a battery lid 5.

The case 1 and the case back 2 are connected by screws 11. An O-ring groove is formed in one part of faces where the case 1 and the case back 2 butt, and an O-ring 12 is disposed in this O-ring groove. Further, the battery lid 5 is fitted to an opening of the battery accommodation chamber in the case back 2. An O-ring groove is formed in one part of the faces where the case back 2 and the battery lid 5 butt, and an O-ring 13 is disposed in this O-ring groove. By these O-ring 12 and O-ring 13, the conventional heartbeat measuring device becomes the waterproof structure.

By the way, before a shipping, the heartbeat measuring device must necessarily undergo an inspection as to whether or not the waterproofness is ensured. The inspection of the conventional heartbeat measuring device, in which the hermetic type waterproof structure has been adopted as mentioned above, has been performed by such a method that a product is put into a water tank for a certain time and an existence/nonexistence of bubble is observed. However, although the waterproofness inspection method like this is simple, there has been a problem that an inspection accuracy is low. Further, after the shipping, if the waterproofness of the heartbeat measuring device decreases, there is a possibility that a sensitivity and an accuracy of the measurement are impaired. Additionally, even if it is normally functioning at present, a possibility that a function decrease is made earlier or it breaks down is high. Whereupon, it is desirable that, not only before the product shipping, the user himself/herself knows the fact that the waterproofness of the heartbeat measuring device is being ensured. This is because, if the fact is understood that there is a problem in the waterproofness, it is possible to suitably take treatments of a use stop and a repair request of the heartbeat measuring device.

A problem that the invention is to solve is to make it possible, in a heartbeat measuring device in which a heartbeat measuring circuit and the like are accommodated in an opaque waterproof case, and which is mounted to a user's chest when used, to simply and certainly perform a check of the waterproofness of the heartbeat measuring device not only before the product shipment but also at the time when a user uses it.

### SUMMARY OF THE INVENTION

In order to solve the problem mentioned above, in the heartbeat measuring device in which the heartbeat measuring circuit and the like are accommodated in the opaque waterproof case, and which is mounted to a user's chest when used, an opening part is provided in one part of the opaque case, and a transparent member is liquid-tightly fixed to the opening part, thereby enabling making it possible to check the waterproofness of the heartbeat measuring device by observing a dew condensation generated on an inner face of the transparent member.

### BRIEF DESCRIPTION OF-THE SEVERAL VIEWS OF THE DRAWINGS

A preferred form of the present invention is illustrated in the accompanying drawings in which:

FIG. 1 is a sectional view showing a heartbeat measuring device of one implementation mode of the invention while being cut in a longitudinal direction;

FIG. 2 is a sectional view showing the heartbeat measuring device of an embodiment 1 of the invention while being cut in the longitudinal direction;

FIG. 3 is a bottom view of the heartbeat measuring device shown with a case back being removed; and

FIG. 4 is a sectional view showing a conventional heartbeat measuring device while being cut in the longitudinal direction.

### DETAILED DESCRIPTION OF THE PREFERRED EMBODIMENTS

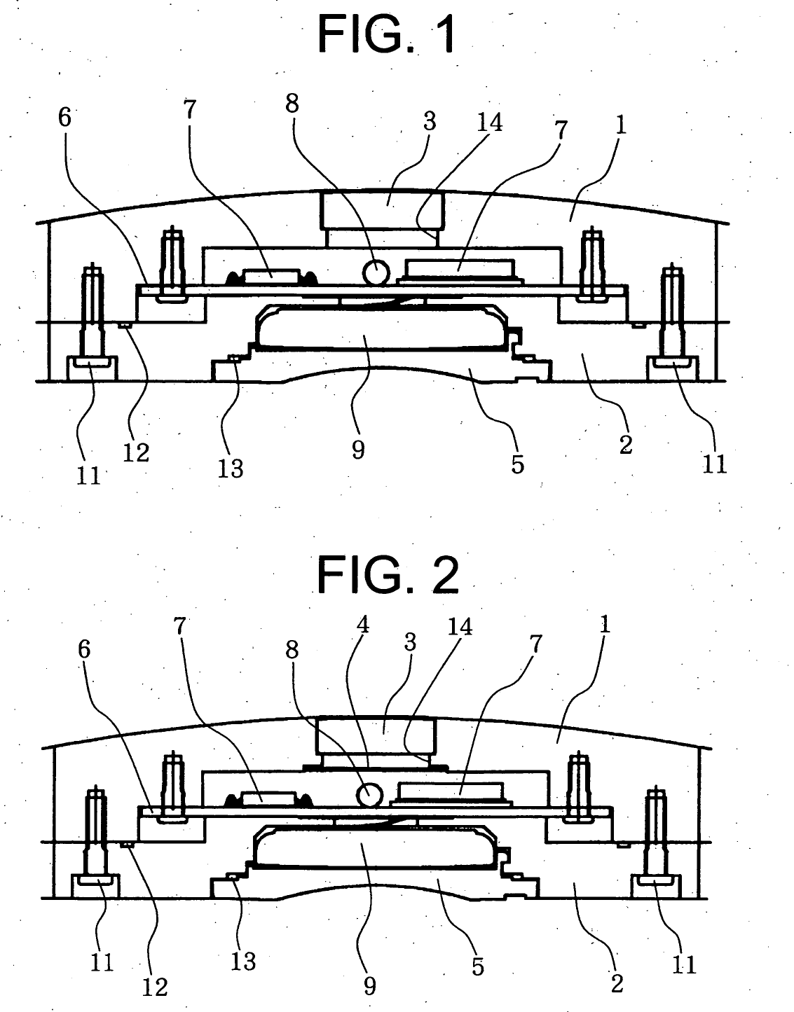

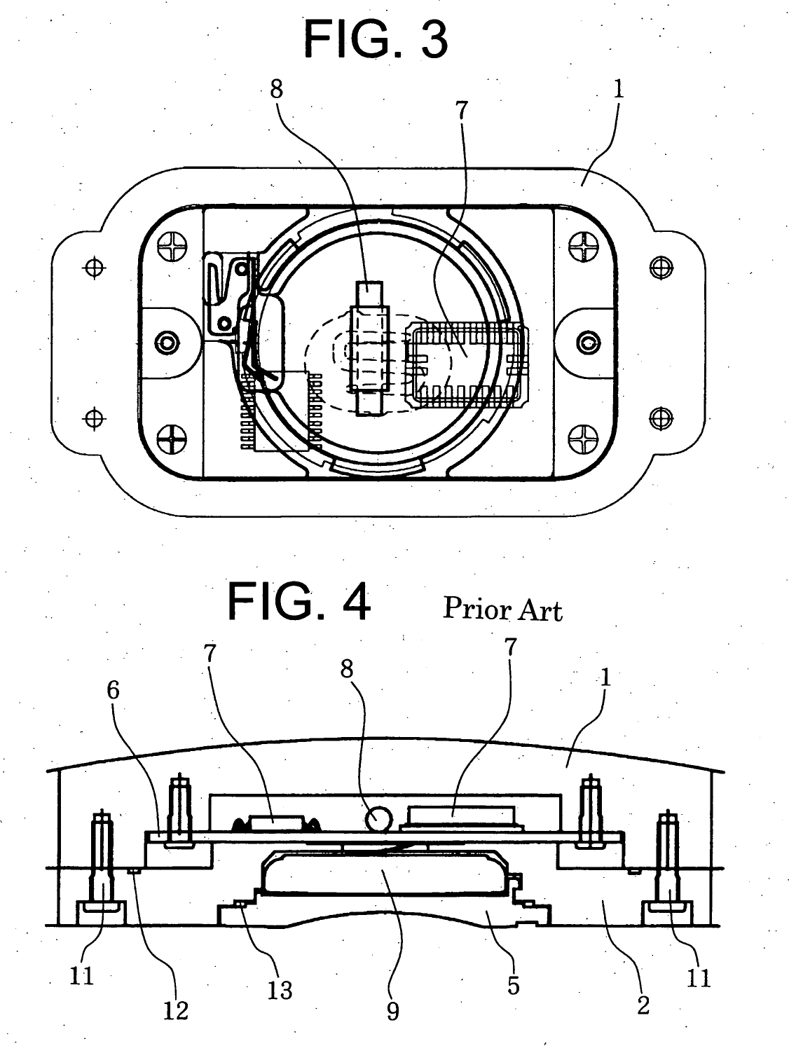

As shown in a sectional view of FIG. 1 shown by being cut in a longitudinal direction and the bottom view of FIG. 3 shown with the case of back being removed, a heartbeat measuring device concerning the invention is configured such that, in a heartbeat measuring device having a case 1 and a case back 2 which are made of opaque hard plastic, a circuit board 6 accommodated in an accommodation chamber formed by the case 1 and the case back 2, various electronic parts 7 attached to the circuit board 6, and an antenna 8, a through-hole 14 is provided in a center part of the opaque case 1, and a transparent member 3 is liquid-tightly fixed to an outside opening part of the through-hole 14, thereby enabling making it possible to check the waterproofness of the heartbeat measuring device by observing the dew condensation generated on the inner face of the transparent member 3.

The through-hole 14 provided in the center part of the opaque case 1 is a cylindrical hole, and its outside opening part has an inner diameter slightly larger than that of the through-hole 14. To this outside opening part, the columnar transparent member 3 is fitted while being caused to have the waterproofness and fixed by an adhesive. It is desirable that the columnar transparent member 3 has such a shape and size that it is entirely fitted to the outside opening part of the through-hole 14. Incidentally, a position where the through-hole 14 is provided may be other position than the center part of the opaque case 1 so long as it is an observable position and, also as to shapes of the transparent member 3 and the through-hole 14, a similar effect is obtained even if they are not limited to a circle.

The circuit board 6 and the various electronic parts 7 constitute the heartbeat measuring circuit, the transmitting circuit and the control circuit. A battery 9 is accommodated in a battery accommodation chamber formed in the case back 2. The opening of the battery accommodation chamber is sealed by a battery lid 5. The case 1 and the case back 2 are connected by screws 11. An O-ring groove is formed in one part of the faces where the case 1 and the case back 2 butt, and an O-ring 12 is disposed in this O-ring groove. Further the O-ring groove is formed in one part of the faces where the case back 2 and the battery lid 10 butt, and an O-ring 13 is disposed in this O-ring groove. By these O-ring 12 and O-ring 13, the heartbeat measuring device concerning the invention becomes the waterproof structure.

In the heartbeat measuring device of an implementation mode mentioned above, the waterproofness check before the shipping of a product is performed as follows. First, the product is previously mounted on a heating plate for a certain time. Next, a waterdrop is dropped on an upper face of the transparent member 3 fixed to the opaque case 1. Thereupon, if moisture exists inside the heartbeat measuring device even in a trace amount, the transparent member 3 clouds. This is because a water vapor evaporated by the heating plate and adhered to the lower face of the transparent member 3 is cooled by the waterdrop dropped on the upper face of the transparent member 3, thereby dew-condensing. Since the heartbeat measuring device in which a cloud like this occurred in the transparent member 3 has no waterproofness, it becomes a failure as the product.

The heartbeat measuring device in which no cloud occurs in the transparent member 3 after passing through such a procedure of the waterproofness check that, after the product mentioned above is mounted on the heating plate for the certain time, the waterdrop is dropped on the upper face of the transparent member 3 fixed to the opaque case 1 is judged that the waterproofness exists.

In the heartbeat measuring device of the implementation mode mentioned above, the user can perform the waterproofness check at the time of use. This is enabled by the fact that, in a case where moisture enters into the inside of the heartbeat measuring device even in the trace amount, this moisture is evaporated due to a high outside air temperature and the like and, just after this if it is exposed to a temperature lower than the above temperature, the cloud occurs in the transparent member 3.

As shown in a sectional view of FIG. 2 shown by being cut in the longitudinal direction and the bottom view of FIG. 3 shown with the case back being removed, an embodiment 1 that is a modified example of the implementation mode of the invention of FIG. 1 is a heartbeat measuring device in which, in the heartbeat measuring device having the case 1 and the case back 2 which are made of the opaque hard plastic, the circuit board 6 accommodated in the accommodation chamber formed by the case 1 and the case back 2, the various electronic parts 7 attached to the circuit board 6, and the antenna 8, the through-hole 14 is provided in the center part of the opaque case 1, the transparent member 3 is liquid-tightly fixed to the outside opening part of the through-hole 14, and additionally an interstice through which the air can move is formed between a lower face of the transparent member 3, thereby disposing a shielding plate 4 to an inside opening part of the through-hole 14. Although the shielding plate 4 is a disc-like opaque plate member, it is provided with plural minute holes in order that the air can flow between the interstice and the accommodation chamber but an inside of the accommodation chamber cannot be seen from an outside.

The inside opening part of the through-hole 14 provided in the center part of the opaque case 1 has an inner diameter slightly larger than that of the through-hole 14. In this inside opening part, the shielding plate 4 of the disc-like opaque plate member is disposed and fixed by the adhesive. Incidentally, in the embodiment mentioned above, in the shielding member 4 of the disc-like opaque plate member, there are provided holes through which the inside of the accommodation chamber cannot be seen from the outside in order to make the flow of the air possible between the interstice and the accommodation chamber, it is also possible to use the shielding plate 4 of the disc-like opaque plate having no holes. In a case where the shielding plate 4 of the disc-like opaque plate having no holes is used, it can be realized by providing, in the case 1, a communication port connecting between the interstice and the accommodation chamber. Or, it can be realized, i.e., also by notching one part of an outer periphery part of the shielding plate 4 of the disc-like opaque plate having no holes.

In the heartbeat measuring device of the embodiment 1 mentioned above, the waterproofness check before the shipment of the product is performed as follows. First, the product is previously mounted on the heating plate for a certain time. Next, the waterdrop is dropped on the upper face of the transparent member 3 fixed to the opaque case 1. Thereupon, if moisture exists inside the heartbeat measuring device even in the trace amount, the transparent member 3 clouds. This is because the water vapor evaporated by the heating plate and adhered to the lower face of the transparent member 3 is cooled by the waterdrop dropped on the upper face of the transparent member 3, thereby dew-condensing. Since the heartbeat measuring device in which the cloud like this has occurred in the transparent member 3 has no waterproofness, it becomes the failure as the product.

The heartbeat measuring device in which no cloud has occurred in the transparent member 3 after passing through such a procedure of the waterproofness check that, after the product mentioned above is mounted on the heating plate for the certain time, the waterdrop is dropped on the upper face of the transparent member 3 fixed to the opaque case 1 is judged that the waterproofness exists.

In the heartbeat measuring device of the embodiment 1 mentioned above, the user can perform the waterproofness check at the time of use. This is because, in the case where moisture enters into the inside of the heartbeat measuring device even in the trace amount, this moisture is evaporated by the high outside air temperature and the like and, just after this if it is exposed to the temperature lower than the above temperature the cloud occurs in the transparent member 3.

Although not shown in the drawing, an embodiment 2 that is other modified example of the implementation mode of the invention of FIG. 1 is configured such that a humidity display member is attached to the lower face of the transparent member 3, and the check of the waterproofness is performed by utilizing a discoloration of the humidity display member. The humidity display member can utilize one marketed as a humidity indicator.

In the heartbeat measuring device of the embodiment 2 mentioned above, the waterproofness check before the shipment of the product is performed as follows. First, the product is mounted on the heating plate for a certain time. Thereupon, if moisture exists inside the heartbeat measuring device even in the trace amount, it adheres to the humidity display member attached to the lower face of the transparent member 3. Since the humidity display member discolors by moisture, it is possible to more easily confirm the fact that moisture exists inside the product. If the humidity display member is made a reversible humidity display member, when it becomes lower than the outside air temperature, the discoloration returns to an original color. Since the heartbeat measuring device in which the discoloration like this of the humidity display member occurred has no waterproofness, it becomes the failure as the product.

The heartbeat measuring device in which, after the product mentioned above was mounted on the heating plate for a certain time, no discoloration occurred in the humidity display member attached to the lower face of the transparent member 3 fixed to the opaque case 1 is judged that the waterproofness exists.

In the heartbeat measuring device of the embodiment 2 mentioned above, the user can perform the waterproofness check at the time of use. This is because, in the case where the moisture has entered into the inside of the heartbeat measuring device even in the trace amount, this moisture is evaporated by the high outside air temperature and the like and adheres to the humidity display member attached to the lower face of the transparent member 3, thereby discoloring the humidity display member.

Although not shown in the drawing, an embodiment 3 that is a modified example of the embodiment of the invention of FIG. 2 is configured such that the shielding plate 4 is constituted by the humidity display member, and the check of the waterproofness is performed by utilizing the discoloration of the humidity display member.

In the heartbeat measuring device of a constitution like this of the invention, if the moisture in the trace amount has entered into the inside of the product, this moisture is evaporated by a temperature higher than the outside air temperature and adheres to the shielding plate 4. Thereupon, since the shielding plate 4 discolors by the moisture, it is possible to more easily confirm the fact that the moisture exists inside the product. If the shielding plate 4 is constituted by the reversible humidity display member, when it becomes lower than the outside air temperature, the discoloration returns to the original color.

The heartbeat measuring device in which, after the product mentioned above is mounted on the heating plate for the certain time, no discoloration occurs in the shielding plate 4 of the humidity display member is judged that the waterproofness exists.

In the heartbeat measuring device of the embodiment 2 mentioned above, the user can perform the waterproofness check at the time of use. This is because, in the case where moisture has entered into the inside of the heartbeat measuring device even in the trace amount, this moisture is evaporated by the high outside air temperature and the like and adheres to the shielding plate 4 of the humidity display member, thereby discoloring the shielding plate.

By the invention, in the heartbeat measuring device in which the heartbeat measuring circuit and the like are accommodated in the opaque waterproof case, and which is mounted to a user's chest when used, it becomes possible that even a user can simply and certainly check the moisture, in a trace amount, having entered into the heartbeat measuring device. Accordingly, if the fact is understood that there is a problem in the waterproofness, a user can suitably take measures such as stopping use of the heartbeat measuring device and requesting a repair thereof.

What is claimed is:

1. A heartbeat measuring device comprising:
an opaque waterproof case to accommodate a heartbeat measuring circuit, which is mounted to a user's chest when used;
wherein a through part is provided in one part of the opaque case, and a transparent member is liquid-tightly fixed to the through part, thereby enabling observing a dew condensation generated on an inner face of the transparent member.

2. A heartbeat-measuring device according to claim 1, wherein the transparent member is liquid-tightly fixed to an outside opening part of the through part.

3. A heartbeat measuring device according to claim 2, further comprising:
a shielding plate which is disposed in an inside opening part of the through part with forming an interstice through which air can move between a lower face of the transparent member.

4. A heartbeat measuring device according to claim 1, further comprising:
a humidity display member which is attached to an inner face of the transparent member.

5. A heartbeat measuring device according to claim 4, wherein the humidity display material is a reversible humidity display material.
